(19) United States
(12) Patent Application Publication
(10) Pub. No.: US 2022/0039793 A1
(43) Pub. Date: Feb. 10, 2022

(54) System And Method For Fixing Sheet-Like Materials To A Target Tissue

(71) Applicant: Cannuflow, Inc., Scotts Valley, CA (US)

(72) Inventor: Theodore R. Kucklick, Scotts Valley, CA (US)

(73) Assignee: Cannuflow, Inc., Scotts Valley, CA (US)

(21) Appl. No.: 17/509,606

(22) Filed: Oct. 25, 2021

Related U.S. Application Data
(63) Continuation of application No. 16/493,126, filed on Sep. 11, 2019.
(60) Provisional application No. 62/472,131, filed on Mar. 16, 2017.

(51) Int. Cl. A61B 17/04

(52) U.S. Cl. CPC A61B 17/0401; A61B 17/0487; A61B 2017/0464; A61B 2017/0417; A61B 2017/0437; A61B 2017/0414

(57) ABSTRACT

The devices, systems and methods herein provide for anchoring of surgical constructs to body tissue. These systems may be used for both biologic implant securement in arthroscopy as well as other sheet and scaffold repair procedures. The system may be used for any soft tissue repair procedure where a synthetic or biologic patch is used, such as joint repair or hernia repair.

This application is a continuation application of U.S. application Ser. No. 16/493,126 filed on Sep. 11, 2019, which is a U.S. National Stage Application of PCT/2018/022968, filed Mar. 16, 2018, which in turn claims priority to U.S. Provisional Application 62/472,131, filed Mar. 16, 2017.

### FIELD OF THE INVENTIONS

The inventions described below relate to the field of arthroscopic repair of joints.

### BACKGROUND OF THE INVENTIONS

Biologic constructs and other sheet-like surgical implants are growing rapidly in popularity for the repair of joint pathologies. Biologic constructs are a family of biologically derived implants to promote tissue growth or to patch and repair tissue defects and tears. These repairs include the repair of arthritic cartilage, the joining of tendons to bone and the bridging of degenerated rotator cuff in the shoulder. Biologic constructs now occupy an increasingly important place in the orthopedic surgeons armamentarium. One of the key problems with biologic constructs is that the attachment systems have not kept pace with advances in these implants. Staples are among the most common form of biologic sheet attachment but there is concern that simple staples may back out and float around loose in the joint.

### SUMMARY

The devices, systems and methods described below provide for anchoring of surgical constructs to body tissue. The systems may be used for both biologic implant securement in arthroscopy as well as other sheet and scaffold repair procedures. The system may be used for any soft tissue repair procedure where a synthetic or biologic patch is used, such as joint repair or hernia repair.

Various implant anchors are described, including a T-bar anchor, a wing shaped anchor and a porcupine anchor. One T-bar anchor uses a strap with teeth that engage a pawl within a locking grommet to secure the sheet implant against the rotator cuff. Another T-bar anchor has an attached suture that connects to a lock button. Another anchor features a sharp end like a curved needle that penetrates the rotator cuff and locks into the tissue when the suture or strap pulls the blunt end backwards. These embodiments securely hold the biologic sheet in place against any suitable tissue such as a rotator cuff, requiring no surgical knot tying, and compress the sheet implant down more securely that a simple staple. The T-bar may go through the rotator cuff or tendon.

The porcupine embodiment is an anchor that has micro molded barbs on the side with a large engaging surface area. This is similar to the way a porcupine quill stays secured in tissue and resists pullout. These microstructured barbs are disposed on a central anchor spike. The head of the anchor comprises a hold-down bar that has additional barbs to hold down the biologic implant sheet.

### BRIEF DESCRIPTION OF THE DRAWINGS

FIGS. 1a, 1b and 1c illustrate a T-Bar implant with locking grommet and an implant driver.

FIGS. 2a and 2b illustrate a lock bar anchor and suture with lock button.

FIGS. 3a, 3b, 3c, 3d and 3e illustrate a T-bar needle delivery device and T-bar implant.

FIGS. 4a, 4b, 4c and 4d illustrate a wing anchor and driver.

FIGS. 5a, 5b and 5c illustrate a porcupine anchor and driver.

### DETAILED DESCRIPTION OF THE INVENTIONS

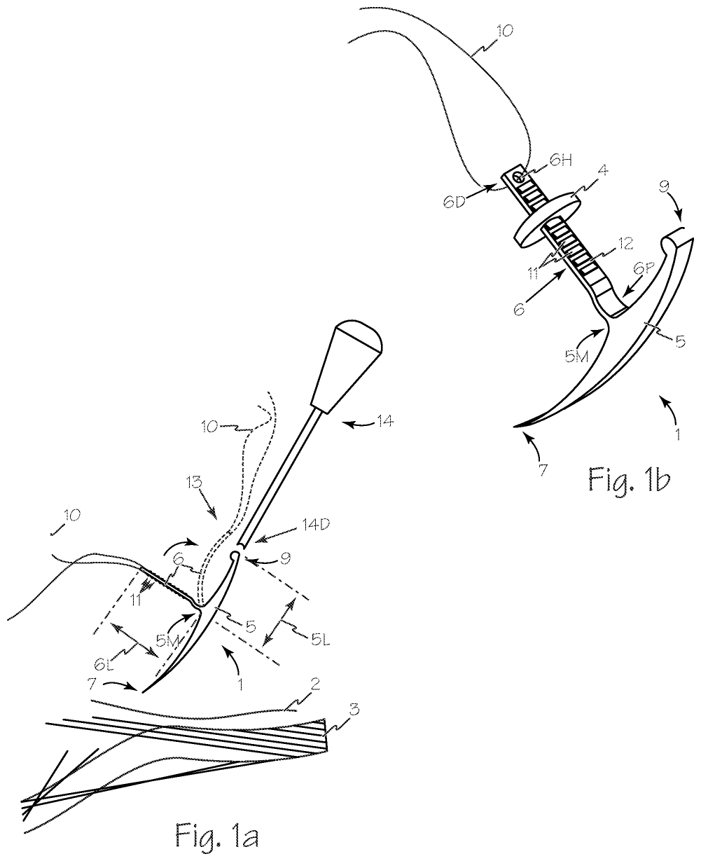

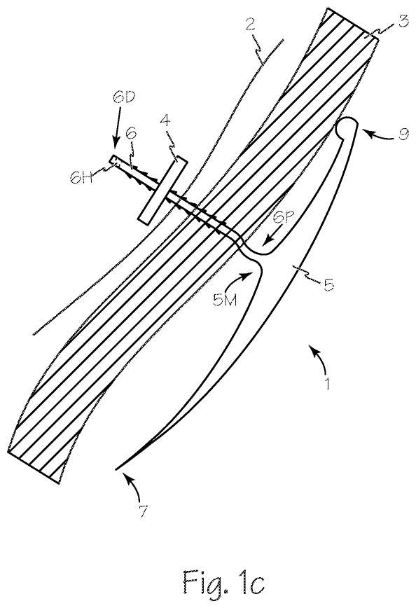

FIGS. 1a, 1b and 1c illustrate a generally T-shaped T-Bar anchor 1 which may be used to secure a sheet like implant such as biological construct 2 to any suitable tissue 3 with a locking grommet 4. The T-bar anchor can also be t-shaped and include an anchor on the bottom of the anchor. The T-Bar anchor comprises implant bar 5 attached at any suitable point such as midpoint 5M to strap 6, thus forming a T-configuration. The implant bar has a sharp end 7 and a blunt or driver end 9. The attachment point is selected to maintain the length 6L of strap 6 to be longer than the distance 5L from the attachment point to the blunt end 9. Strap 6 has a keyhole 6H in distal end 6D for inserting a guide suture such as suture 10. Proximal end 6P of the strap is attached to implant bar 5 at any suitable location between the sharp end and the blunt end such as midpoint 5M. Strap 6 has teeth 11 along one or more sides such as side 12 and the teeth engage locking grommet 4 as in a ratchet. The teeth engage any suitable element within the locking grommet such as a pawl or the interior surface of the locking grommet.

In use, an implant sheet is apposed to tissue at a suitable treatment site. For example, FIG. 1a shows a biologic construct 2 apposing rotator cuff tissue 3. The user pulls the guide suture 10 toward blunt end 9 of implant bar 5, which in turn bends or folds strap 6 parallel to the implant bar as illustrated in folded position 13 of FIG. 1a. The user then holds T-bar anchor 1 with the pointed end of the implant bar pointed at implant sheet. Implant driver 14 shown has a distal tip 14D which engages the bar implant blunt end 9. Implant driver 14 is used to push the blunt end of the implant bar, thus driving the sharp end of the implant bar through implant sheet 2 and rotator cuff tissue 3. Once implant bar 5 has reached a desirable position, the user withdraws the driver which allows the implant bar to return to a T-configuration with strap 6 perpendicular to implant bar 5 with the distal end 6D of the strap accessible through the tissue and implant sheet. The user retaining control over guide suture 10 provides insurance against having the entirety of T-bar anchor 1 pass through the implant sheet and the tissue and be unable to be secured with a locking grommet. If necessary, when the strap ends up below sheet 2 and or tissue 3 the user will pull suture 10 to draw the strap back out of the perforation created during insertion of the T-bar through the biological construct and the tissue. Locking grommet 4 is then pushed over distal end 6D of the strap and the lock grommet is pushed along the strap toward the proximal end of the strap engaging teeth 11 to secure the sheet implant against the rotator cuff tissue. Once installed, the perpendicular orientation of implant bar 5 relative to strap 6 results in a generally T-shaped T-bar anchor 1.

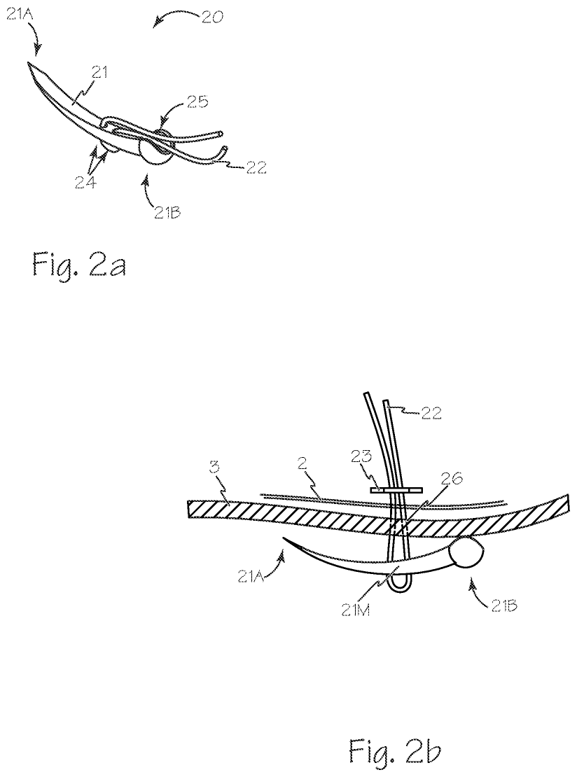

FIGS. 2a and 2b illustrate a lock bar anchor 20 which comprises lock bar 21, suture 22 and lock button 23. Lock bar 21 has a sharp penetrating end 21A and a blunt drive end 21B. The bar has one or two holes 24 in any suitable location such as mid point 21M to engage suture 22 in loop fashion. Suture 22 is fed through one hole 24 and back out through the other hole 24 as shown, so that it may draw the bar toward the lock button securing implant sheet 2 apposing tissue 3 as illustrated in FIG. 2b.

In use, the implant sheet 2 is positioned apposing tissue 3 at any suitable treatment site. For example, FIG. 2b shows biologic sheet 2 apposing rotator cuff tissue 3. The user extends suture 22 flat along the lock bar 21 with the suture extending through indent or channel 25 in blunt drive end 21B. The user holds lock bar 21 with pointed end 21A pointing to securement location 26 through implant sheet 2 and drives the lock bar anchor through the implant sheet and the tissue until drive end 21B passes through tissue 3. When all of lock bar 21 passes through tissue 3 the lock bar is allow to rotate until it is perpendicular to suture 22. The user then pulls the sutures taught and inserts the sutures through a lock button to draw the bar and lock button together, thus securing the sheet to the rotator cuff. Once installed, the perpendicular orientation of lock bar 21 relative to suture 22 results in a generally T-shaped lock bar anchor 20.

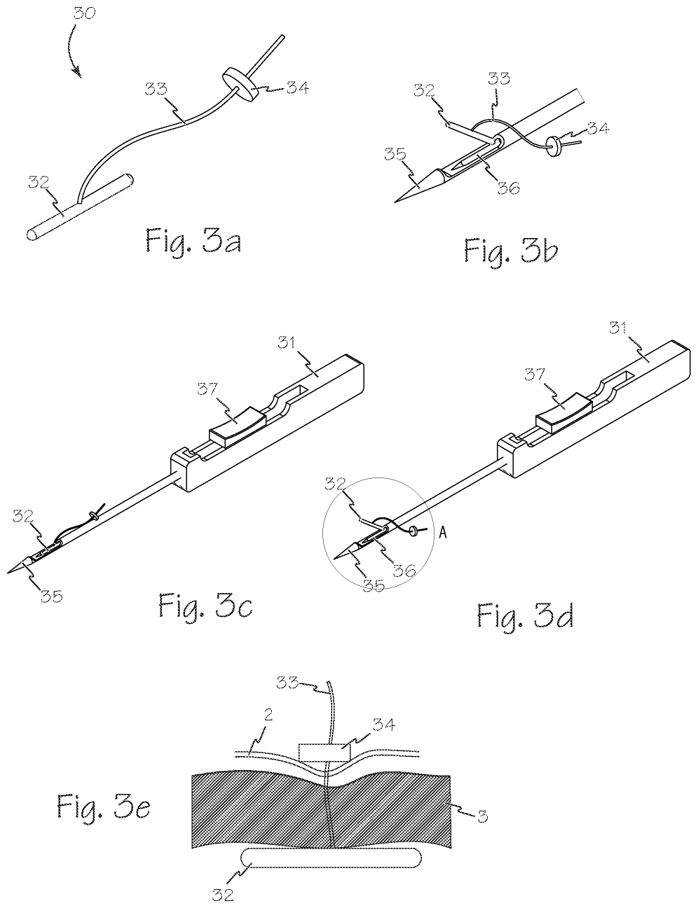

FIGS. 3a, 3b, 3c, 3d and 3e illustrate a T-bar implant anchor 30, driver 31 and the T-bar anchor secured to tissue 3. T-bar 32 has a suture such as suture 33 secured at the longitudinal center of the T-bar to form the T-bar implant. A sliding lock grommet such as grommet 34 is slid down suture 33 to secure an implant sheet to any suitable tissue such as tissue 3. T-bar driver 31 has a sharp distal end 35 and a hollow space 36 for holding the T-bar implant just proximal to the sharp distal end of the T-bar driver. The T-bar driver device has a sliding bar 37 operably connected to the handle for ejecting or driving the T-bar implant into the surgical space.

In use, biologic sheet 2 is positioned apposing tissue 3 at any suitable treatment site. For example, FIG. 3e shows biologic sheet 2 apposing tendon 3. The user loads the T-bar implant into T-bar driver 31 as shown in FIGS. 3b and 3c. The user slidably engages the delivery device slide 37 to drive the T-bar implant through the biologic sheet and tendon. The user withdraws the T-bar driver leaving T-bar 32 positioned under the tendon with suture 33 attached and extending through the tendon and sheet. The user pulls suture 33 through lock grommet 34 to draw the T-bar and lock grommet together, thereby securing the sheet to the tendon. Once installed, the perpendicular orientation of T-bar implant 32 relative to suture 33 results in a generally T-shaped T-bar anchor 30.

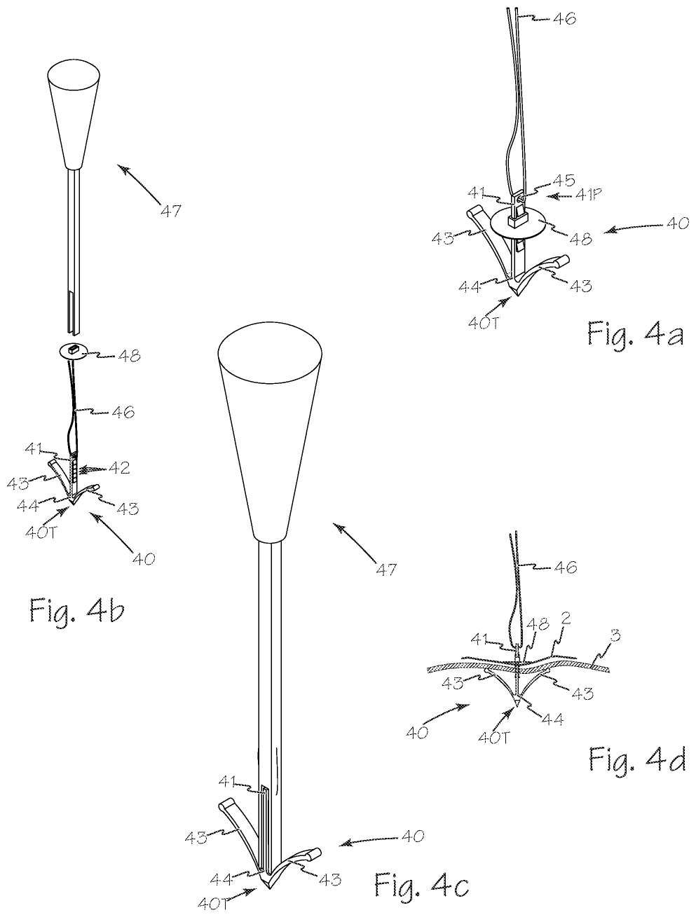

FIGS. 4a, 4b, 4c and 4d illustrate a generally T-shaped wing anchor 40 as well as accessories and a driver. Wing anchor 40 has a center post, post 41 with locking ratchet teeth such as teeth 42. Two wings, wings 43, resiliently spring outwardly and extend from center post 41 at its distal end, post root 44. The orientation of wings 43 relative to center post 41 results in generally T-shaped wing anchor 40. Center post 41 has a keyhole, hole 45 positioned at its proximal end 41P for engaging suture 46. Anchor driver 47 has a hollow portion 47H at its distal end sized and dimensioned to engage wing anchor 40 at post root 44 and enclose post 41 as shown in FIG. 4c. Wing anchor 40 also includes an obturator tip 40T for penetrating implant sheets and tissue.

In use, biologic sheet 2 is positioned apposing tissue 3 at any suitable treatment site. For example, FIG. 4d shows implant sheet 2 apposing rotator cuff 3. The user inserts the suture 46 through hole 45 and holds the suture off to the side. The user holds wing anchor 40 with the pointed obturator tip 40T over the implant sheet. The driver hollow portion is inserted over the wing anchor center post. The user pushes the driver to drive the wing anchor through the implant sheet and tissue 3 into the securement site. Once the wing anchor has fully penetrated tissue 3 the wings will spring away from the driver and post. The user removes the driver while still holding on to the suture. The suture is fed through lock grommet 48 and the lock grommet is pushed distally on the center post to sandwich the lock grommet against the implant sheet and retain the implant sheet in contact with the tissue.

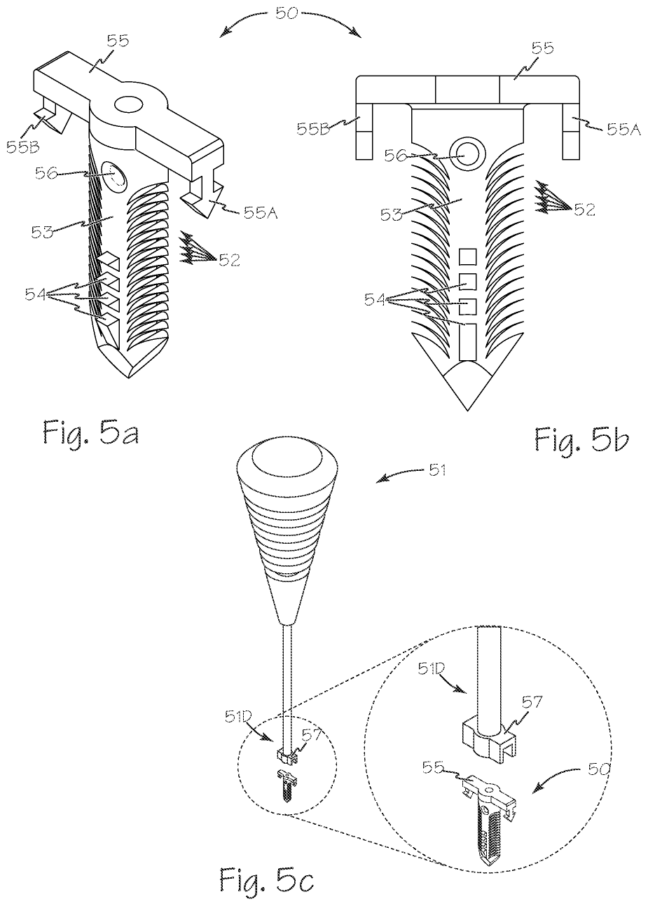

FIGS. 5a, 5b and 5c illustrate a generally T-shaped porcupine anchor 50 and driver 51. Flexible “porcupine” or microstructured barbs such as barbs 52 are disposed on a central anchor spike 53 along with fixed ratchet teeth 54. The head of anchor 50 comprises a hold-down bar 55, which has side stakes 55A and 55B at opposite ends of the hold-down bar. The combination of central anchor spike 53 and hold-down bar 55 result in a generally T-shaped porcupine anchor 50. Central anchor spike 53 also includes a suture channel or keyhole 56 in the proximal end for a guide suture. Driver 51 is used to push the porcupine anchor into position through any suitable implant sheet to engage tissue with the flexible porcupine quills and the ratchet teeth engaging the tissue and resisting pullout. The distal end of the driver, distal end 51D includes anchor mating end 57 sized and dimensioned to frictionally engage hold-down bar 55.

While the preferred embodiments of the devices and methods have been described in reference to the environment in which they were developed, they are merely illustrative of the principles of the inventions. The elements of the various embodiments may be incorporated into each of the other species to obtain the benefits of those elements in combination with such other species, and the various beneficial features may be employed in embodiments alone or in combination with each other. Other embodiments and configurations may be devised without departing from the spirit of the inventions and the scope of the appended claims.

I claim:

1. An implant anchor comprising:
a generally t-shaped body having a central post having a proximal end and a sharp distal end, the proximal end perpendicularly secured to a bar, said bar characterized by first and second sharp side stakes at opposite ends of the bar;
a plurality of flexible curved microstructure barbs disposed along the entire length of the central post; and
a keyhole at the proximal end of the central post for insertion of a guide suture.

2. The implant anchor of claim 1 further comprising a plurality of ratchet teeth disposed along the central post.

3. The implant anchor of claim 1 wherein the keyhole comprises a suture channel.

4. A system for delivering biological constructs to a target site within a patient comprising:
an implant anchor including:
a generally t-shaped body having a central post having a proximal end and a sharp distal end, the proximal end perpendicularly secured to a bar, said bar characterized by first and second sharp side stakes at opposite ends of the bar;
a plurality of flexible curved microstructure barbs disposed along the entire length of the central post; and
a keyhole at the proximal end of the central post for insertion of a guide suture; and
a driver having a proximal and distal end wherein the distal end is sized and dimensioned to engage the bar of the implant anchor.

5. The system of claim 4 wherein the implant anchor further comprises a plurality of ratchet teeth disposed along the central post.

6. The system of claim 4 wherein the keyhole of the implant anchor comprises a suture channel.
